(19) United States
(12) Patent Application Publication
(10) Pub. No.: US 2025/0319155 A1
(43) Pub. Date: Oct. 16, 2025

(54) PREPARATION OF TOLERIZING NANOPARTICLES FOR THE TREATMENT OF PEANUT ALLERGY

(71) Applicant: COUR PHARMACEUTICALS DEVELOPMENT COMPANY INC., Skokie, IL (US)

(72) Inventors: John Puisis, Skokie, IL (US); Michael Boyne, Skokie, IL (US); Samantha Reilly, Skokie, IL (US); Greta Wodarcyk, Skokie, IL (US); Qichen Xu, Skokie, IL (US); Zhiyi Lin, Skokie, IL (US); Timothy Toby, Skokie, IL (US)

(21) Appl. No.: 18/713,321

(22) PCT Filed: Nov. 23, 2022
(86) PCT No.: PCT/US2022/080409

§ 371 Date: May 24, 2024

Related U.S. Application Data
(60) Provisional application No. 63/282,889, filed on Nov. 24, 2021.

(51) Int. Cl. A61K 38/16; A61K 9/51

(52) U.S. Cl. CPC A61K 38/168; A61K 9/5123; A61K 9/5138; A61K 9/5153; A61K 9/5192

(57) ABSTRACT

The present disclosure relates to a process for the preparation of tolerizing immune modifying nanoparticles encapsulating peanut proteins, compositions comprising the particles and use thereof for the treatment of peanut allergy.

### CROSS-REFERENCE TO RELATED APPLICATIONS

The present application is a national phase of PCT/US2022/080409, filed Nov. 23, 2022 which claims the priority benefit of U.S. Provisional Patent Application No. 63/282,889, filed Nov. 24, 2021, herein incorporated by reference in its entirety.

### FIELD OF THE DISCLOSURE

The present disclosure relates to the process for the preparation of tolerizing immune modifying nanoparticles encapsulating peanut proteins for the treatment of peanut allergy.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 27,276 bytes, file named “57037_Seqlisting.hml”; created on Nov. 15, 2022.

### BACKGROUND

Peanut allergy is one of the most common food allergies affecting nearly 1.2% of the total US population and 2.5% of the pediatric population with incidence rates on the rise over the past decade.¹ Peanut allergy is driven by a pathologic hyperimmune response where exposure to peanut can lead to mild to severe symptoms such as nausea, vomiting, rashes, impaired breathing, drop in blood pressure, and even death.

The allergic immune response to peanut proteins is mediated by T-cell dependent mechanisms involving upregulation of T helper type-2 responses, B cell class switching leading to production of peanut protein specific IgE antibodies, and degranulation of mast cells and basophils.²

Currently, there is no cure for peanut allergy with strict avoidance of exposure to peanut antigens and management of anaphylaxis the only options available to patients. Immune tolerizing therapies which can induce T-cell tolerance to allergenic peanut proteins are considered the gold standard for the treatment of peanut allergy; however, such therapies have been elusive.

Attempts at developing immune tolerizing therapies have been made using oral immunotherapy (OIT), subcutaneous immunotherapy (SCIT), epicutaneous immunotherapy (EPIT), and sublingual immunotherapy (SLIT).³ The success of these approaches has been highly variable and only desensitization to peanut proteins has been reported, which offers protection against only accidental exposure but is not a cure.⁴⁻⁶ Moreover, these approaches rely on chronic administration of formulations containing free peanut proteins. As a result, these therapies pose a risk of adverse reactions, including anaphylaxis, in peanut allergic patients due to exposure of free allergenic peanut proteins to an immune system with pre-existing sensitivity to these allergens.⁶⁻⁷

### SUMMARY

Tolerizing immune modifying particles (TIMPs), comprising one or more antigens, have been previously described for the treatment of immune-mediated disorders (e.g., autoimmune diseases and allergies) via induction of antigen-specific immune tolerance (WO2013192532 and WO2015023796 incorporated herein by reference). Encapsulation of peanut proteins within TIMP core is an advantage as it ensures safe delivery of encapsulated proteins to APCs without inducing immune activation (e.g., by exposure to IgE) reducing the risk of adverse reactions (e.g., anaphylaxis) associated with administration of free peanut proteins in peanut allergic patients.

The process for manufacturing of TIMP-PPE involves numerous steps each of which influences the physiochemical properties of resulting composition essential for safe and therapeutic administration. Importantly, the process must be optimized to ensure efficient encapsulation of peanut proteins within the particle core.

The present disclosure provides a process for manufacturing a composition comprising negatively charged particles encapsulating peanut proteins (TIMP-PPE). The process is directed to a process of manufacturing particles optimized for safe and therapeutic administration of TIMP-PPE for the treatment of peanut allergy. In various embodiments, the method comprises: (a) generating a primary emulsion by mixing an aqueous solution of peanut proteins (PPE) with an oil phase including a polymer; (b) mixing the primary emulsion with a solution including one or more surfactants and/or stabilizers to form a secondary emulsion; (c) hardening the secondary emulsion by evaporation to remove the solvent resulting in hardened polymeric nanoparticles encapsulating peanut proteins within their cores; (d) filtering, washing, and concentrating the nanoparticles; and (e) freeze drying the nanoparticles. In various embodiments, the primary emulsion of step (a) is a water-in-oil emulsion. In various embodiments, the secondary emulsion of step (b) is an oil-in-water emulsion.

In various embodiments, the aqueous solution of step (a) includes a solvent. In various embodiments, the solvent is an organic solvent. In various embodiments, the solvent is an inorganic solvent. In various embodiments, the organic solvent is dichloromethane, acetone, ethanol, methylene chloride, dimethyl sulfoxide (DMSO), ethyl acetate, dimethylformamide, tetrahydrofuran, chloroform, and acetic acid. In various embodiments, the inorganic solvent is water, ammonia, sulphuric acid, carbon disulphide, bromine trifluoride, phosphorous oxychloride, hydrogen fluoride, and sulphur dioxide. In various embodiments, the solvent in the aqueous solution is at a concentration of 1, 2, 3, 5, 10, 15, 20, 25, 30, 35, 40, 45, or 50% (v/v). In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 mM. In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 M.

In various embodiments, the surfactant and/or stabilizer solution of step (b) includes a solvent. In various embodiments, the solvent is an organic solvent. In various embodiments, the solvent is an inorganic solvent. In various embodiments, the organic solvent is dichloromethane, acetone, ethanol, methylene chloride, dimethyl sulfoxide (DMSO), ethyl acetate, dimethylformamide, tetrahydrofuran, chloroform, and acetic acid. In various embodiments, the inorganic solvent is water, ammonia, sulphuric acid, carbon disulphide, bromine trifluoride, phosphorous oxychloride, hydrogen fluoride, and sulphur dioxide. In various embodiments, the solvent in the aqueous solution is at a concentration of 1, 2, 3, 5, 10, 15, 20, 25, 30, 35, 40, 45, or 50% (v/v). In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 mM. In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 M. In various embodiments, the solvent in the solution of step (a) and step (b) are the same. In various embodiments, the solvents in the solution of step (a) and step (b) are different.

In various embodiments, the aqueous solution of step (a) includes 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, or 100 mg/mL peanut proteins. In various embodiments, the peanut protein is dissolved in the aqueous solution by mixing for 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, peanut proteins used in the process of manufacturing TIMP-PPE are obtained from roasted peanuts. In various embodiments, the peanut proteins are obtained from raw peanut. In various embodiments, the peanut proteins for use in the process of manufacturing TIMP-PPE are obtained using a method comprising: (a) grinding raw peanuts into a paste; (b) defatting the peanut paste; (c) drying the defatted peanut paste; (d) powdering the dried peanut paste; (e) extracting peanut protein from the peanut powder using ammonium bicarbonate; and (f) concentrating and clarifying the peanut protein resulting in purified peanut extract. In various embodiments, the purified peanut extract is further purified to isolate allergenic peanut proteins. In various embodiments, the isolated allergenic peanut proteins are obtained by fractionation. In various embodiments, the allergenic peanut proteins are Ara h 1, Ara h 2, Ara h 3, Ara h 4, Ara h 5, Ara h6, Ara h 7, Ara h 8, Ara h 9, Ara h 10, Ara h 11, Ara h 12, Ara h 13, Ara h 14, Ara h15, Ara h 16, Ara h 17 and Ara h 18. In various embodiments, the aqueous solution of step (a) contains peptides from allergenic peanut proteins. In various embodiments, the peptides comprise allergenic epitopes from allergenic peanut proteins Ara h 1, Ara h 2, Ara h 3, Ara h 4, Ara h 5, Ara h6, Ara h 7, Ara h 8, Ara h 9, Ara h 10, Ara h 11, Ara h 12, Ara h 13, Ara h 14, Ara h15, Ara h 16, Ara h 17 and Ara h 18. In various embodiments, the peptides are obtained from naturally occurring peanut proteins. In various embodiments, the peptides are manufactured synthetically. In various embodiments, the peptides are manufactured by solid phase peptide synthesis or solution phase peptide synthesis.

In various embodiments the purified peanut extract used in step (a) is dissolved in a solvent. In various embodiments the solvent is an organic solvent. In various embodiments the solvent is an inorganic solvent. In various embodiments the purified peanut extract used in step (a) is dissolved in an inorganic solvent that comprises one or more acids and/or one or more bases. In various embodiments the solvent has a pH between 1.0 and 14.0. In various embodiments the pH is 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0 including all values lying within this range. In various embodiments the solvent is acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid, barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia, aluminium hydroxide, sodium carbonate, magnesium hydroxide, zinc hydroxide, ferrous hydroxide, acetone, lithium hydroxide, pyridine, rubidium hydroxide. In various embodiments the solvent concentration is between 0.01% to 100% (v/v). In various embodiments the concentration of the solvent is about 0.01%, about 0.05%, about 0.1%, about 0.2%, about 0.3%, about 0.4%, about 0.5%, about 0.6%, about 0.7%, about 0.8%, about 0.9%, about 1%, about 10%, about 25%, about 50%, about 75%, about 99%, about 100% (v/v) including all values lying within this range. In various embodiments the solvent concentration is between 0.1 M to 36 M. In various embodiments the solvent concentration is about 0.1M, about 0.5 M, about 1 M, about 2 M, about 3 M, about 4 M, about 5 M, about 6 M, about 7 M, about 8 M, about 9 M, about 10 M, about 11 M, about 12 M, about 13 M, about 14 M, about 15 M, about 16 M, about 17 M, about 18 M, about 20 M, about 30 M, about 36 M including all values lying within this range. In various embodiments, the concentration of the dissolved purified peanut extract is about 0.1, about 0.2, about 0.5, about 1, about 2, about 3, about 4, about 5, about 6, about 7, about 8, about 9, about 10, about 11, about 12, about 13, about 14, about 15, about 20, about 25, about 30, about 35, about 40, about 45, about 50, about 55, about 60, about 65, about 70, about 75, about 80, about 85, about 90, about 95, or about 100 mg/mL including all values lying within this range. In various embodiments, the purified peanut extract is dissolved in the solvent by mixing for 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours.

In various embodiments, the dissolved peanut protein extract has a pH between 1.0 and pH 6 including all values lying within this range. In various embodiments the dissolved peanut protein extract has a pH from about pH 1 to about pH 6, from about pH 2 to about pH 6, from about pH 3 to about pH 6, from about pH 2 to about pH 4, or about pH 1, about pH 1.5, about pH 2, about pH 2.5, about pH 3, about pH 3.5, about pH 4, about pH 4.5, about pH 5, about pH 5.5, or about pH 6.

In various embodiments, the polymer in step (a) is a biodegradable polymer. In various embodiments, the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid. In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0.

In various embodiments, the polymer in step (a) is PLGA. In various embodiments, the molar ratio of co-polymers of PLGA are 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In various embodiments, the PLGA has a high molecular weight. In various embodiments, the PLGA has a low molecular weight. In various embodiments, the PLGA has a molecular weight of between 10 to 10,000 kDa (e.g., between 10, 20, 30, 40, 50, 60, 70, 80, 90, 100, 500, 1000, 2000, 3000, 4000, 5000, 6000, 7000, 8000, 9000, or 10000 kDa including all values lying within this range). In various embodiments, the amount of PLGA in the solution of step (a) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight.

In various embodiments, the surfactant and/or stabilizer used in step (b) is anionic, cationic, or nonionic. In various embodiments, the surfactant and/or stabilizer is a poloxamer, a polyamine, polyethylene glycol (PEG), Tween-80, gelatin, dextran, pluronic L-63, pluronic F-68, pluronic 188, pluronic F-127, polyvinyl alcohol (PVA), polyacrylic acid (PAA), methylcellulose, lecithin, didodecyldimethylammonium bromide (DMAB), poly(ethylene-alt-maleic acid) (PEMA), vitamin E TPGS (D-a-tocopheryl polyethylene glycol 1000 succinate), hyaluronic acid, poly amino acids (e.g., polymers of lysine, arginine, aspartic acid, glutamic acid, serine, threonine, tyrosine and cysteine, or their enantiomers), methylcellulose, hydroxyethylcellulose, hydroxyprolylcellulose, hydroxypropylmethylcellulose, gelatin, sodium cholate, a carbomer, or a sulfate polymer (e.g., heparin sulfate, chondroitin sulfate, fucoidan, ulvan, and carrageenan). In various embodiments, the amount of surfactant and/or stabilizer present in the solution in step (b) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the surfactant and/or stabilizer have a molecular weight of between 0.1 to 10,000 kDa (e.g., between 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 500, 1000, 2000, 3000, 4000, 5000, 6000, 7000, 8000, 9000, or 10000 kDa including all values lying within this range).

In various embodiments, the solution including one or more surfactants and/or stabilizers that form an oil-in-water secondary emulsion in (b) has a pH of about 4 or less than about 4.0. In various embodiments, the oil-in-water secondary emulsion has a pH of about pH 1 to about pH 4, about pH 2 to about pH 4, about pH 3 to about pH 4, or about pH 1, about pH 1.5, about pH 2, about pH 2.5, about pH 3, about pH 3.5, or about pH 4.

In various embodiments, the method comprises: (a) generating a primary emulsion by mixing aqueous solution of peanut proteins with an oil phase including a polymer resulting in a water-in-oil primary emulsion; (b) mixing the primary emulsion with a solution including one or more surfactants and/or stabilizers to form an oil-in-water secondary emulsion; (c) hardening the secondary emulsion to remove the solvent resulting in polymeric nanoparticles encapsulating PPE within their cores; (d) filtering, washing, and concentrating the nanoparticles; and (e) freeze drying the nanoparticles.

In various embodiments, the water-in-oil primary emulsion of step (a) is obtained by homogenization of the aqueous solution of peanut proteins with the oil phase including a polymer. In various embodiments, homogenization is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the oil-in-water secondary emulsion of step (b) is obtained by homogenization of the primary emulsion with a solution including one or more surfactants and/or stabilizer. In various embodiments, homogenization is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the water-in-oil primary emulsion of step (a) is obtained by sonication of the aqueous solution of peanut proteins with the oil phase including a polymer. In various embodiments, sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds. In various embodiments, the oil-in-water secondary emulsion of step (b) is obtained by sonication of the primary emulsion with a solution including one or more surfactants and/or stabilizers. In various embodiments, sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds

In various embodiments, the secondary emulsion is hardened by evaporation. In various embodiments, the evaporation is active evaporation. In various embodiments, the evaporation is passive evaporation. In various embodiments, the active evaporation is vacuum-driven evaporation. In various embodiments, evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, the secondary emulsion is hardened by evaporation. In various embodiments, the evaporation is active evaporation. In various embodiments, the evaporation is passive evaporation. In various embodiments, the active evaporation is performed using stirring or under vacuum. In various embodiments, the active evaporation is performed under high-pressure vacuum. In various embodiments, the active evaporation is performed under low pressure vacuum. In various embodiments, evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, the evaporation is performed at a pressure of between 0.01 and 1000 mBar (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 mBar including all including all values lying within this range).

In various embodiments, the filtration, washing, and concentration of particles in step (d) is performed by gel filtration, membrane filtration, dialysis, centrifugation, chromatography, density gradient centrifugation, or combinations thereof.

The present disclosure also contemplates a process for manufacturing a composition comprising negatively charged TIMPs encapsulating peanut proteins (TIMP-PPE). In various embodiments, TIMP-PPE particles have a negative zeta potential. In various embodiments, the negative zeta potential of TIMP-PPE particles is between about −100 mV to about 0 mV. In various embodiments, the zeta potential of the particles is from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

In various embodiments, the size, or diameter, of TIMP-PPE particles is between 0.05 μm to about 10 μm. In various embodiments, the diameter of TIMP-PPE particles is between 0.1 μm and about 10 μm. In various embodiments, the diameter of TIMP-PPE particles is between 0.1 μm and about 5 μm. In various embodiments, the diameter of TIMP-PPE particles is between 0.1 μm and about 3 μm. In various embodiments, the diameter of TIMP-PPE particles is between 0.3 μm and about 5 μm. In various embodiments, the diameter of TIMP-PPE particles is about 0.3 μm to about 3 μm. In various embodiments, the diameter of TIMP-PPE particles is between about 0.3 μm to about 1 μm. In various embodiments, the diameter of TIMP-PPE particles is between about 0.4 μm to about 1 μm. In various embodiments, the TIMP-PPE particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PPE particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm. In various embodiments, the polydispersity index (PDI) or heterogeneity index for particle size is between 0.01 and 1.0 (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 including all values within the range).

In various embodiments, the particles have a homogenous size distribution. In various embodiments, the particles have a homogenous size distribution wherein at least 90% of the particles have a diameter of between 0.05 μm and about 10, between 0.1 μm and about 10, 0.1 μm and about 5, 0.1 μm and about 3, 0.3 μm and about 5, 0.3 μm to about 3 μm. In various embodiments, the particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PPE particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PPE particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PPE particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm.

In various embodiments, the invention of the present disclosure provides a process for manufacturing a composition comprising negatively charged particles encapsulating peanut proteins (TIMP-PPE). In various embodiments, the peanut protein content encapsulated within the TIMP-PPE composition is 0.1 to 100 ug/mg. In various embodiments, the peanut protein content is between 0.1 to 100 ug/mg (e.g., 0.1, 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, or 100 ug/mg) including all values and ranges that lie in between these values. In various embodiments, the peanut proteins encapsulated within the TIMP-PPE composition include Ara h proteins. In various embodiments, the Ara h proteins are Ara h 1, Ara h 2, Ara h 3, Ara h 4, Ara h 5, Ara h6, Ara h 7, Ara h 8, Ara h 9, Ara h 10, Ara h 11, Ara h 12, Ara h 13, Ara h 14, Ara h15, Ara h 16, Ara h 17, and Ara h 18. In various embodiments, the content of any one of or combinations of the Ara h proteins in the TIMP-PPE composition is between 0.01 to 100 ug/mg (e.g., 0.01, 0.02, 0.03, 0.04, 0.05, 0.06, 0.07, 0.08, 0.09, 0.1, 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, or 100 ug/mg including all values and ranges that lie in between these values). In various embodiments, the process of making TIMP-PPE as described herein yields an encapsulation efficiency between 1-100% (e.g., 1, 2, 3, 4, 5, 6, 7, 8, 9, 10, 15, 20, 25, 30, 40, 50, 60, 70, 80, 90, or 100% including all values and ranges that lie between these values). In various embodiments, the process yields an encapsulation efficiency of at least 20%. The peanut protein content in the TIMP-PPE composition can be determined by methods described in the literature including ELISA, Mass Spectrometry, HPLC, CBQCA, and Western Blot.

In various embodiments, the present disclosure provides a process for manufacturing a composition comprising negatively charged particles encapsulating peanut proteins (TIMP-PPE), wherein the particle surface contains low levels of peanut proteins. In various embodiments, the particle surface is essentially free of peanut proteins. In various embodiments, the amount of peanut proteins present on the surface of the particles is between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) of the total protein content of the TIMP-PPE composition. In various embodiments, the frequency of particles containing peanut proteins on their surface is between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) higher compared to a negative control. In various embodiments, the frequency of particles containing peanut proteins on their surface is 0-100% % (e.g., 0, 1, 2, 3, 4, 5, 6, 7, 8, 9, 10, 20, 30, 40, 50, 60, 70, 80, 90, and 100% including all values and ranges that lie between these values) lower when compared to a positive control. In various embodiments, the amount of peanut proteins on the surface of the particles is between 0-10-fold (e.g., 0, 1, 2, 3, 4, 5, 6, 7, 8, 9, or 10 fold including all values and ranges that lie between these values) higher than a negative control. In various embodiments, the amount of peanut proteins on the surface of the particles is between 0-100-fold (e.g., 0, 1, 2, 3, 4, 5, 6, 7, 8, 9,10, 20, 30, 40, 50, 60, 70, 80, 90, or 100-fold including all values and ranges that lie between these values) lower than a positive control. In various embodiments, the number of TIMP-PPE particles with peanut proteins on their surface is determined using previously described methods such as flow cytometry, Mass Spectrometry, ELISA, CBQCA, and Western Blot.

In various embodiments, the present disclosure provides a process for manufacturing a composition comprising negatively charged particles encapsulating peanut proteins (TIMP-PPE), wherein the particles exhibit low burst release. In various embodiments, the particles exhibit no burst release. In various embodiments, the particle burst release is between 0-75% (e.g., 0, 1, 2, 3, 4, 5, 6, 7, 8, 9, 10, 20, 30, 40, 50, 60, 70, or 75% including all values and ranges that lie between these values).

In various embodiments, excipients are added to the nanoparticle composition prior to freeze drying in step (e). In various embodiments, the excipients are buffering agents and/or cryoprotectants. In various embodiments, the excipients are selected from the group consisting of sucrose, mannitol, trehalose, sorbitol, dextran, Ficoll, Dextran 70 k, sodium citrate, lactose, L-arginine, or glycine. In various embodiments, the amounts of excipients added to the nanoparticle composition prior to freeze drying is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the amounts of excipients added to the nanoparticle composition prior to freeze drying is between 0.01 and 500 g (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, or 500 g) per gram of nanoparticles.

In various embodiments, the manufacturing batch sizes of TIMP-PPE can be scaled up or down. In various embodiments, the manufacturing batch size is between 0.01 g to 100 kg. In various embodiments, the batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg or 100 kg including all values and ranges that lie between these values.

Contemplated herein is a particle encapsulating peanut proteins made by the methods described herein. Also provided is a composition comprising particles encapsulating peanut proteins made by the methods described herein. In various embodiments, the composition further comprises a pharmaceutically acceptable carrier, diluent or excipient. In various embodiments, the pharmaceutical composition is a sterile pharmaceutical composition.

Also provided is a formulation comprising a particle comprising peanut protein extract. In various embodiments, formulations or pharmaceutical compositions of TIMP-PPE contain negatively charged particles encapsulating purified protein extract, and excipients. In various embodiments, the excipients are selected from the group consisting of sucrose, mannitol, trehalose, sorbitol, dextran, Ficoll, Dextran 70 k, sodium citrate, lactose, L-arginine, or glycine. In various embodiments TIMP-PPE formulations contain between one to eleven excipients. In various embodiments, TIMP-PPE formulations contain one or more, two or more, three or more, four or more, five or more, six or more, seven or more, eight or more, nine or more, or ten or more excipients.

In various embodiments, TIMP-PPE formulations contain negatively charged particles encapsulating purified peanut protein, sucrose, mannitol, and sodium citrate. In various embodiments, the negatively charged particle concentration in the TIMP-PPE formulation is between 1 to 100%, between 20 to 50%, or between 30 to 40%, including all ranges and values that lie between these ranges. In various embodiments, the negatively charged particle concentration in the TIMP-PPE formulation is about 30%, about 31%, about 32%, about 33%, about 34%, about 35%, about 35.6%, about 36%, about 37%, about 38%, about 39%, or about 40%.

In various embodiments, the sucrose concentration in the TIMP-PPE formulation is between 1 to 100%, between 20 to 50%, or between 30 to 40%, including all ranges and values that lie between these ranges. In various embodiments, the sucrose concentration in the TIMP-PPE formulation is about 30%, about 31%, about 32%, about 33%, about 34%, about 35%, about 35.6%, about 36%, about 37%, about 38%, about 39%, or about 40%.

In various embodiments, the mannitol concentration in the TIMP-PPE formulation is between 1 to 100%, between 15 to 35%, or between 20 to 30%, including all ranges and values that lie between these ranges. In various embodiments, the sucrose concentration in the TIMP-PPE formulation is about 20%, about 21%, about 22%, about 23%, about 24%, about 25%, about 25%, about 26%, about 26.7%, about 27%, about 28%, about 29%, or about 30%.

In various embodiments, the sodium citrate concentration is between 0.01 to 25% or between 0.5 to 3.5%, including all ranges and values that lie between these ranges. In various embodiments, the sodium citrate concentration is about 0.5%, about 1%, about 1.5%, about 2%, about 2.1%, about 2.5%, about 3%, or about 3.5%.

In various embodiments, the purified peanut protein in the TIMP-PPE formulation is between 0.3 μg to 30 μg (micrograms) peanut protein per milligram (mg) of PLGA, or between 1 μg to 10 μg peanut protein per mg PLGA, including all ranges and values that lie between these ranges. In various embodiments, the purified peanut protein in the TIMP-PPE formulation is about 1 μg, about 2 μg, about 3 μg, about 4 μg, about 5 μg, about 6 μg, about 7 μg, about 8 μg, about 9 μg, or about 10 μg peanut protein per mg of PLGA.

The disclosure provides for methods of treating peanut allergy in a subject comprising administering to the subject particles encapsulating peanut proteins as described herein. Also contemplated is a composition comprising TIMP-PPE as described herein for use in treating peanut allergy. In various embodiments, the disclosure provides for use of a composition comprising TIMP-PPE as described herein in the preparation of a medicament for treating peanut allergy.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIG. 1. Characterization of PPE by SDS-PAGE. PPE was separated by electrophoretic mobility in non-reducing conditions and stained to visualize protein banding patter. A standard molecular weight size reference was run in parallel. The results of the characterization by SDS-PAGE positively identified Ara h1, h2, h3, and h6 band groupings at the expected size.

FIG. 2. High-level manufacturing process flow diagram depicting the main steps involved in the manufacture of tolerizing nanoparticles encapsulating peanut proteins (TIMP-PPE).

FIG. 3. Physiochemical characterization of TIMP-PPE particles manufactured in a 80 g batch using Scanning Electron Microscopy (SEM). Shown is a representative SEM image of nanoparticles at 10,000× magnification.

FIG. 4. Physiochemical characterization of TIMP-PPE particles manufactured in a 160 g batch using Scanning Electron Microscopy (SEM). Shown is a representative SEM image of nanoparticles at 10,000× magnification.

### DETAILED DESCRIPTION

TIMPs are surface functionalized negatively charged poly(lactide-co-glycolide) particles encapsulating antigenic proteins or peptide epitopes associated with inflammatory conditions such as autoimmune diseases and allergies. TIMPs are designed for targeted delivery of encapsulated proteins/peptides to antigen presenting cells (APCs) of the mononuclear phagocyte system resulting in APC mediated T cell reprogramming via non-inflammatory pathways.

In pre-clinical models of autoimmune diseases and allergies, TIMPs have demonstrated therapeutic efficacy at inducing T-cell tolerance to antigenic/allergenic proteins and peptides resulting in improved disease symptoms.⁸⁻¹² TIMPs encapsulating peanut proteins (TIMP-PPE) can potentially treat peanut allergies by reprogramming the immune system and inducing antigen specific T cell tolerance to peanut proteins. There is a current need for immune tolerizing therapies which can induce T-cell tolerance to allergenic peanut proteins for long term therapeutic benefit without exposing patients to risk of adverse events.

The present disclosure provides a process for manufacturing negatively charged particles encapsulating peanut proteins (TIMP-PPE) and pharmaceutical compositions comprising the particles.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term ‘particle’ may be used to encompass beads and spheres.

“Negatively charged particle” as used herein refers to particles which have been modified to possess a net surface charge that is less than zero.

“Surface-functionalized” as used herein refers to particles which have one or more functional groups on its surface. In some embodiments, the surface functionalization occurs by the introduction of one or more functional groups to a surface of a particle. In various embodiments, surface functionalization may be achieved by carboxylation (i.e., addition of one or more carboxyl groups to the particle surface) or addition of other chemical groups (e.g., other chemical groups that impart a negative surface charge).

“Carboxylated particles” or “carboxylated beads” or “carboxylated spheres” includes any particle that has been modified or surface functionalized to add one or more carboxyl group onto the particle surface. Carboxylation of the particles can be achieved using any compound which adds carboxyl groups, including, but not limited to, Poly(ethylene-maleic anhydride) (PEMA), Poly(acrylic acid), or a poly amino acid consisting of carboxyl side-chains (e.g., aspartic acid, glutamic acid). Carboxylation may also be achieved by using polymers with native carboxyl groups (e.g., PLGA) to form particles, in which the manufacturing process results in additional carboxyl groups, i.e., in addition to those naturally expressed by the polymer, being located on the surface of the particle.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via hosts cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration) or via inhalation.

The term “pharmaceutically acceptable” or “pharmacologically acceptable” refers to material that is not biologically or otherwise undesirable, i.e., the material may be administered to an individual without causing any undesirable biological effects or without interacting in a deleterious manner with any of the components of the composition in which it is contained or with any components present on or in the body of the individual.

The term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure that is identical to the epitope used to generate the antibody yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotope. “Epitope” as used herein is also known as an “antigenic determinant”, which is the part of an antigen that is recognized by the immune system, specifically by antibodies, B cells, or T cells. For example, an epitope is a specific piece of the antigen that an antibody binds to. The part of an antibody that binds to the epitope is called a paratope. Although epitopes are usually non-self-proteins, sequences derived from the host that can be recognized (as in the case of autoimmune diseases) are also epitopes. T cell epitopes are presented on the surface of an antigen-presenting cell, where they are bound to MHC (major histocompatibility complex) molecules. In humans, professional antigen-presenting cells are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids

The terms “treat”, “treated”, “treating” and “treatment”, as used with respect to methods herein refer to eliminating, reducing, suppressing or ameliorating, either temporarily or permanently, either partially or completely, one or more clinical symptom, manifestation or progression of an event, disease or condition. Such treating need not be absolute to be useful. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### Particles

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a negative charge of between 0 to about −100 mV. In various embodiments, the particles are 400-800 nanometers in diameter and have a charge of between about −25 mV and −70 mV. In various embodiments, the particles are 400-1000 nanometers in diameter and have a charge of between about −25 mV and −70 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PGA particles, PLA particles, PLGA particles, PLURONICS stabilized polypropylene sulfide particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, citrate, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22:1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0, or about 20:80 to 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), or a poly(lactic-co-glycolic acid) particle, or a carboxylated polyglycolic acid particle (PGA), carboxylated polylactic acid particle (PLA), or carboxylated poly(lactic-co-glycolic acid) particle. In certain embodiments, the particle has a copolymer ratio of polylactic acid/polyglycolic acid 80:20: polylactic acid/polyglycolic acid 90:10: or polylactic acid/polyglycolic acid 50:50. In various embodiments, the particle is a poly(lactic-co-glycolic acid) particle and has a copolymer ratio of about 50:50 polylactic acid: polyglycolic acid.

It is contemplated that the particle may further comprise a surfactant and/or stabilizer. The surfactant and/or stabilizer can be anionic, cationic, or nonionic. Surfactants in the poloxamer and polaxamine family are commonly used in particle synthesis. Surfactants and/or stabilizers that may be used, include, but are not limited to PEG, Tween-80, gelatin, dextran, pluronic L-63, PVA, PAA, methylcellulose, lecithin, DMAB and PEMA. Additionally, biodegradable and biocompatible surfactants including, but not limited to, vitamin E TPGS (D-α-tocopheryl polyethylene glycol 1000 succinate), poly amino acids (e.g., polymers of lysine, arginine, aspartic acid, glutamic acid, serine, threonine, tyrosine and cysteine, or their enantiomers), sodium cholate and sulfate polymers. In certain embodiments, two surfactants are used. In certain embodiments, two stabilizers are used. In certain embodiments, a combination of two or more surfactants and stabilizers are used. For example, if the particle is produced by a double emulsion method, the two surfactants can include a hydrophobic surfactant for the first emulsion, and a hydrophobic surfactant for the second emulsion. For example, stabilizers can be compounds which stabilize the primary and/or the secondary emulsion as described herein by providing a physical barrier or an energy barrier between adjacent nanoparticle droplets in the emulsion, thereby reducing their probability to coalesce and form larger nanoparticle droplets.

In certain embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference (Sánchez et al., Molecules 25 (16): 3760, 2020; Zielińska et al. Molecules. 25 (16): 3731, 2020).

### Antigens

An antigen refers to a discreet portion of a molecule, such as a polypeptide or peptide sequence, a 3-D structural formation of a polypeptide or peptide, a polysaccharide or polynucleotide that can be recognized by a host immune cell. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more peanut antigens. The one or more peanut antigens are derived from peanut protein extract or may be peptides derived from known peanut proteins, e.g., isolated from the protein or made synthetically.

Over 15 peanut allergens are recognized to date by the WHO/IUIS Allergen Nomenclature Sub-Committee (database maintained at www.allergen.org), Ara h1 to Ara h18. Peanut allergens can be classified into different groups based on their architecture (e.g., trimer, monomer, cupin, albumin, prolamin, profilin, oleosins, defensins, vincillin, Nonspecific lipid transfer proteins (nsLTPs)) based on Ara h1, h2, h3, h5, h6 and h8, and each of these groups possesses a different degree of allergenic potency (Ozias-Akins et al., Allergy 74:888-898, 2019). Known peanut allergens include those derived from Arachis hypogaea Ara h1, Ara h2, Ara h3, Ara h5, Ara h6, Ara h7, and Ara h8. See e.g., UNIPROT E5G076 showing the Ara h1 polypeptide sequence (SEQ ID NO: 1), UNIPROT A0A445BY15 for Ara h2 polypeptide (SEQ ID NO: 2), UNIPROT E5G077 for Ara h3 polypeptide (SEQ ID NO: 3) (see also UNIPROT Database No. 082580 (SEQ ID NO: 4) and Q9SQH7 (SEQ ID NO: 5) for Ara h3 isoallergens 1 and 2 (formerly Ara h4), respectively), UNIPROT L7QH52 for Ara h5 polypeptide (SEQ ID NO: 6), UNIPROT A5Z1RO for Ara h6 polypeptide (SEQ ID NO: 7), UNIPROT B4XID4 for Ara h7 polypeptide (SEQ ID NO: 8), or UNIPROT Q6VT83 for Ara h8 polypeptide sequence SEQ ID NO 9), Ara h9, isoallergen1 and 2, UNIPROT Database No. B6CEX8 and B6CG41, (SEQ ID NO: 10 and 11) respectively; Ara h10, isoallergen 1 and 2, UNIPROT Database No. Q647G5 and Q647G4, (SEQ ID NO: 12 and 13) respectively; Ara h11, isoallergen 1 and 2, UNIPROT Database No. Q45W87 and Q45W86, (SEQ ID NO: 14 and 15) respectively; Ara h12 UNIPROT Database No. B3EWP3 (SEQ ID NO: 16); Ara h13, isoallergen 1 and 2, UNIPROT Database No. B3EWP4 and COHJZ1, (SEQ ID NO: 17 and 18) respectively; Ara h14, isoallergen 1, 2, and 3, UNIPROT Database No. Q9AXI1, Q9AXIO and Q6J1J8, (SEQ ID NO: 19-21) respectively; Ara h15, UNIPROT Database No. Q647G3 (SEQ ID NO: 22); Ara h16, UNIPROT Database No. A0A509ZX51 (SEQ ID NO: 23); Ara h17, UNIPROT A Database No. 0A510A9S3 (SEQ ID NO: 24); and Ara h18, UNIPROT Database No. A0A444XS96 (SEQ ID NO: 25).

In certain embodiments, one, two, three, or a higher number of antigens or antigenic peptides are used in the TIMPs. In certain embodiments, the one or more peanut antigens is encapsulated in the TIMP by covalent linkage to the interior surface of the particle (See e.g., US Patent Publication US20190282707, herein incorporated by reference). In certain embodiments, it is contemplated that sequences of two or more peanut proteins, e.g., from Ara h1, Ara h2, Ara h3, Ara h5, Ara h6, Ara h7, and/or Ara h8, are linked in a fusion protein and encapsulated within a TIMP described herein. Methods for making TIMP with linked epitopes are described in US Patent Publication US20190365656, herein incorporated by reference.

Emulsions occur in many forms of processing and are used extensively by the food, cosmetics and drug delivery. Oil-water (single) or water-oil-water (double) emulsion are methods by which PLGA can be used to encapsulate hydrophobic and hydrophilic drugs in micro- or nanoscale form. In summary, PLGA is dissolved into an organic phase (oil) that is emulsified with a surfactant or stabilizer (water). Hydrophobic drugs and/or other agents are added directly to the oil phase, whereas hydrophilic drugs and/or other agents (water) may be first emulsified with the polymer solution prior to formation of particles. High intensity homogenization (e.g., sonication bursts) facilitate the formation of small polymer droplets. The resulting emulsion is added to a larger aqueous phase and stirred for several hours, which allows the solvent to evaporate. Hardened nanoparticles are collected and washed by centrifugation. In certain embodiments, hardened emulsion particles can be obtained through evaporation of the oil phase.

“Water-in-oil-in-water” (W/O/W) emulsion is an example of a double emulsion, in which dispersions of small water droplets within larger oil droplets are themselves dispersed in a continuous aqueous phase. Because of their compartmentalized internal structure, double emulsions can provide advantages over simple oil-in-water emulsions for encapsulation, such as the ability to carry both polar and non-polar cargos (pharmaceutical/biological agent, e.g., proteins), and improved control over release of therapeutic molecules. The preparation of double emulsions typically requires surfactants or their mixtures for stability. The surfactants stabilize droplets subjected to extreme flow, leading to direct, mass production of robust double nanoemulsions that are amenable to nanostructured encapsulation applications in various industries. In one example, a double emulsion process involves generating a primary emulsion by mixing an aqueous solution of a pharmaceutical/biological agent(s) with a solution including a polymer resulting in a water-in-oil primary emulsion. The primary emulsion is then mixed with a solution including one or more surfactants to form an oil-in-water secondary emulsion. The secondary emulsion is then hardened by evaporation to remove the solvent(s) resulting in hardened polymeric nanoparticles encapsulating the pharmaceutical/biological agent(s).

“Homogenization” as used herein relates to an operation using a class of processing equipment referred to as homogenizers that are geared towards reducing the size of droplets in liquid-liquid dispersions. Factors that affect the particle or droplet size include but are not limited to the type of emulsifier, emulsifier concentration, solution conditions, and mechanical device (homogenizing power; pressure, rotation speed, time). Non-limiting examples of homogenizers include high speed blender, high pressure homogenizers, colloid mill, high shear dispersers, ultrasonic disruptor membrane homogenizers, and ultrasonicators. Mechanical homogenizers, manual homogenizers, sonicators, mixer mills, vortexers, and the like may be utilized for mechanical and physical disruption within the scope of the disclosure.

“Batch size” as used herein relates to the scale of manufacture depending on the weight of the final product. The manufacturing process can be altered, scaled up or scaled down. The manufacturing process can be altered, scaled up or down by altering the amount or volume of the solvent, antigens/proteins, polymer, surfactants, stabilizers, cryoprotectants or excipients. The manufacturing process can be scaled up or down by altering the time of homogenization, sonication, evaporation, filtration, concentration, washing or lyophilization.

Methods for determining protein content in the particles or in solution include ELISA, Mass Spectrometry, HPLC, CBQCA, and Western Blot.

Molecular Probes CBQCA Protein Quantitation Kit provides a rapid and highly sensitive method for the quantitation of proteins in solution. The kit utilizes the ATTO-TAG CBQCA reagent (3-(4-carboxybenzoyl) quinoline-2-carboxaldehyde) originally developed as a chromatographic derivatization reagent for amines. This reagent has also proven extremely useful for quantitating amines in solution, including the accessible amines in proteins. The ATTO-TAG CBQCA reagent is virtually non-fluorescent in aqueous solution; however, in the presence of cyanide, it reacts with primary amines such as those found in proteins to form highly fluorescent derivatives.

### Pharmaceutical Formulations

Pharmaceutical compositions of the present disclosure containing the TIMP-PPE described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcohol-ic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the particles are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-PPE described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Formulations comprising a particle comprising peanut protein extract and excipients are provided. Exemplary excipients include sucrose, mannitol, trehalose, sorbitol, dextran, Ficoll, Dextran 70 k, sodium citrate, lactose, L-arginine, or glycine. In various embodiments TIMP-PPE formulations contain between one to eleven excipients. In various embodiments, TIMP-PPE formulations contain one or more, two or more, three or more, four or more, five or more, six or more, seven or more, eight or more, nine or more, or ten or more excipients.

In some embodiments, TIMP-PPE formulations contain negatively charged particles encapsulating purified peanut protein, sucrose, mannitol, and sodium citrate. In various embodiments, the negatively charged particle concentration in the TIMP-PPE formulation is between 1 to 100%, between 20 to 50%, or between 30 to 40%, including all ranges and values that lie between these ranges. In various embodiments, the negatively charged particle concentration in the TIMP-PPE formulation is about 30%, about 31%, about 32%, about 33%, about 34%, about 35%, about 35.6%, about 36%, about 37%, about 38%, about 39%, or about 40%.

In various embodiments, the sucrose concentration in the TIMP-PPE formulation is between 1 to 100%, between 20 to 50%, or between 30 to 40%, including all ranges and values that lie between these ranges. In various embodiments, the sucrose concentration in the TIMP-PPE formulation is about 30%, about 31%, about 32%, about 33%, about 34%, about 35%, about 35.6%, about 36%, about 37%, about 38%, about 39%, or about 40%.

In various embodiments, the mannitol concentration in the TIMP-PPE formulation is between 1 to 100%, between 15 to 35%, or between 20 to 30%, including all ranges and values that lie between these ranges. In various embodiments, the sucrose concentration in the TIMP-PPE formulation is about 20%, about 21%, about 22%, about 23%, about 24%, about 25%, about 25%, about 26%, about 26.7%, about 27%, about 28%, about 29%, or about 30%.

In various embodiments, the sodium citrate concentration is between 0.01 to 25% or between 0.5 to 3.5%, including all ranges and values that lie between these ranges. In various embodiments, the sodium citrate concentration is about 0.5%, about 1%, about 1.5%, about 2%, about 2.1%, about 2.5%, about 3%, or about 3.5%.

In various embodiments, the purified peanut protein in the TIMP-PPE formulation is between 0.3 μg to 30 μg (micrograms) peanut protein per milligram (mg) of PLGA, or between 1 μg to 10 μg peanut protein per mg PLGA, including all ranges and values that lie between these ranges. In various embodiments, the purified peanut protein in the TIMP-PPE formulation is about 1 μg, about 2 μg, about 3 μg, about 4 μg, about 5 μg, about 6 μg, about 7 μg, about 8 μg, about 9 μg, or about 10 μg peanut protein per mg of PLGA.

### Methods of Use

Provided herein is a method of treating peanut allergy in a subject comprising administering to the subject TIMP-PPE described herein, wherein TIMP-PPE is administered at a dose of 0.001 to 12 mg/kg or 0.1 to 12 mg/kg. Also provided herein is a method of reducing an allergic immune response to peanut antigens in a subject suffering from peanut allergy comprising administering to the subject TIMP-PPE, wherein TIMP-PPE is administered at a dose of 0.001 to 12 mg/kg or 0.1 to 12 mg/kg.

Also contemplated, the TIMP-PPE is administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. Optionally, the TIMP-PPE is administered in a dose of about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. Alternatively, TIMP-PPE is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In another embodiment, TIMP-PPE is administered at a concentration of between about 0.0005 mg/mL and about 50 mg/mL or about 0.05 mg/mL and about 50 mg/mL, optionally about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/ml, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

It is contemplated that the TIMP-PPE is administered in a single dose or in multiple doses. In various embodiments, TIMP-PPE is administered once weekly, once every two weeks, once every three weeks, once every 4 weeks, once every two months, once every three months, once every 6 months, or once per year. In certain embodiments, TIMP-PPE is administered in two doses one-week apart.

In various embodiments, TIMP-PPE is administered intravenously, subcutaneously, intramuscularly, intraperitoneally, intranasally, or orally. It is contemplated that if TIMP-PPE is given intravenously, it can be via intravenous infusion lasting about 1, 2, 3, 4, 5, 6, 7, 8, 10, 12, 18 or 20 hours.

It is further contemplated that TIMP-PPE is administered alone or in combination with one or more additional therapeutics. Exemplary additional therapeutics include, but are not limited to, inhibitors of IgE, inhibitors of basophil activation, inhibitors of mast cell activation, an antihistamine, nonsteroid anti-inflammatory drugs (NSAID), or a small molecule or biological therapeutic.

It is provided that administering TIMP-PPE to a subject in need thereof, alone or in combination with one or more additional therapeutics, relieves one or more symptoms of peanut allergy. Symptoms of peanut allergy include skin reactions, hives, skin redness, skin swelling, itching, tightening of the throat, difficulty breathing, shortness of breath, and anaphylaxis.

It is also contemplated that administering TIMP-PPE to a subject in need thereof, alone or in combination one or more additional therapeutics, reduces the duration and severity of an allergic immune response to peanut proteins or following exposure to peanut proteins. An allergic immune response contemplated herein includes a Th2 T cell response, B-cell activation, basophil activation, eosinophil activation, mast cell activation, and/or IgE induction.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

In a further embodiment, the disclosure provides an article of manufacture, or unit dose form, comprising: (a) a composition of matter comprising TIMP-PPE as described herein; (b) a container containing said composition; and (c) a label affixed to said container, or a package insert included in said container referring to the use of said TIMP-PPE in the treatment of peanut allergy as described herein.

Additional aspects and details of the disclosure will be apparent from the following examples, which are intended to be illustrative rather than limiting

### EXAMPLES

### Example 1. Process for Preparing Purified Peanut Extract for the Manufacture of Tolerizing Nanoparticles Encapsulating Peanut Proteins (TIMP-PPE)

One process of PPE manufacture utilizes raw peanut (Arachis hypogaea) which can be sourced from a commercial vendor. Briefly, the process begins by grinding the raw peanut into a fine paste. The ground peanut is then defatted by acetone extraction. The defatted peanut material is allowed to air dry for 1-5 days and is then heat dried for 12-24 hours. The dried material is powdered by placing it through a sieve with a mesh ≤2.0 mm. Protein is then extracted from the powdered peanut raw material with ammonium bicarbonate, separated by centrifugation, and clarified by 1 μm filtration. The final solution is concentrated/dialyzed using a hollow fiber cartridge, re-centrifuged, and clarified.

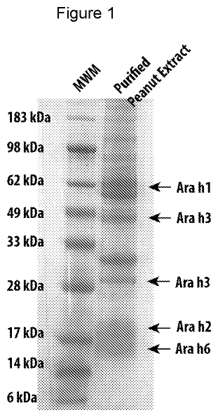

Prior to use in the manufacture of TIMP-PPE, peanut protein extract was characterized to confirm the presence of antigenic peanut proteins (e.g., Ara h 1, Ara h 2, Ara h 3, Ara h 4, Ara h 5, Ara h6, Ara h 7, Ara h 8, Ara h 9, Ara h 10, Ara h 11, Ara h 12, Ara h 13, Ara h 14, Ara h15, Ara h 16, Ara h 17 and Ara h 18) by SDS-PAGE and total protein content by CBQCA. Results of SDS-PAGE assay are shown in FIG. 1. Protein content in the peanut protein extract was determined to be 0.73 mg/mL.

### Example 2. Process for Manufacturing Tolerizing Nanoparticles Encapsulating Peanut Proteins (TIMP-PPE)

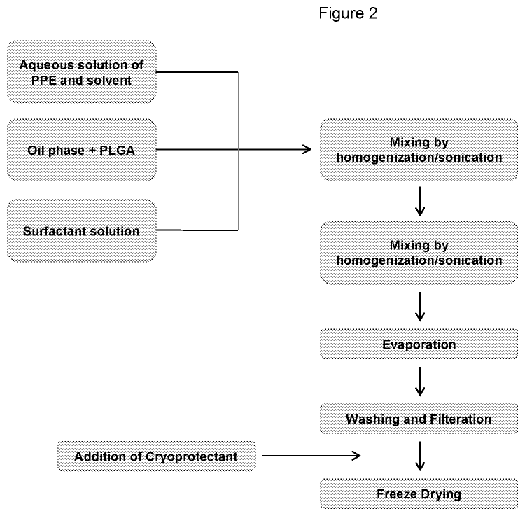

TIMP-PPE (CNP-201) was manufactured using a double-emulsion solvent evaporation process. A high-level manufacturing process flow diagram is shown in FIG. 2. Briefly, purified peanut extract dissolved in 1M acetic acid (10 mg/mL) was rapidly mixed with a 5% PLGA solution (50:50; molecular weight between 10,000 to 60,000 Da) in ethyl acetate to generate a primary water-in-oil emulsion. The primary emulsion was then rapidly mixed with a surfactant and stabilizer solution containing 4% PVA and PAA dissolved (Sigma Aldrich, 100 KDa, 35% wt) in ethyl acetate to form an oil-in-water secondary emulsion. The composition of the PVA/PAA/Ethyl acetate blend is maintained at a pH below 4.0. Mixing of the primary and secondary emulsions was performed by homogenization.

Solvent was removed from the secondary emulsion by evaporation under pressure for a total of at least 3-4 hours. Hardened nanoparticles were then washed in sterile water and concentrated by filtration using a 20 μm filter. Cryoprotectants sucrose and mannitol and buffering agent sodium citrate dihydrate were added to the hardened nanoparticles. The formulation was then lyophilized.

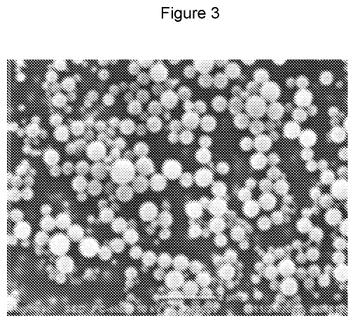

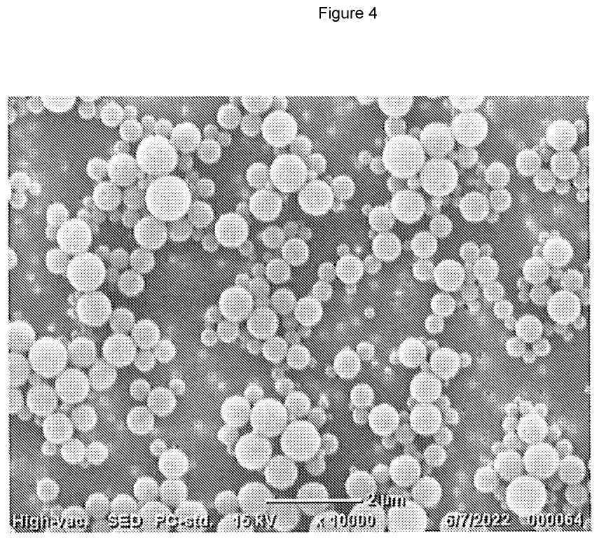

The final CNP-201 formulation was characterized to determine physiochemical properties such as particle diameter, zeta potential, total protein content, Ara h protein presence, burst release, and presence of proteins on the surface of the particles. The results of CNP-201 characterization, manufactured at a 80 g batch size or at a 160 g batch size are provided in Table 1 and Table 2. CNP-201 particles were examined by Scanning Electron Microscopy showing a homogenous composition of intact particles with smooth surfaces manufactured at an 80 g batch size (FIG. 3) and at a 160 g batch size (FIG. 4).

TABLE 1
Physiochemical characterization of CNP-201 particles encapsulating
purified peanut extract manufactured at an 80 g batch size.

| Nanoparticle Physiochemical Property | Result | |
|---|---|---|
| Particle Size by Dynamic Light Scattering | 500 | nm |
| Zeta Potential by Dynamic Light | −49 | mV |
| Scattering | | |
| Particle Size by Laser Diffraction | 992 | nm |
| Ara h ELISA | Ara h1, h2, h3, and h6 present | |
| Protein Content by CBQCA | 7.4 | μg/mg |
| Protein content by Peanut ELISA | 1.1 | μg/mg |
| Burst Release by CBQCA | 36.6% | |
| Particle Size Distribution by SPOS | 520/580/710 | nm |
| (d₁₀/d₅₀/d₉₀) | | |
| Surface protein by Flow Cytometry | ≤25% positive | |
| Endotoxin | <0.048 | EU/mg |

TABLE 2
Physiochemical characterization of CNP-201 particles encapsulating
purified peanut extract manufactured at a 160 g batch.

| Nanoparticle Physiochemical Property | Result | |
|---|---|---|
| Particle Size by Dynamic Light Scattering | 510 | nm |
| Zeta Potential by Dynamic Light | −51 | mV |
| Scattering | | |
| Particle Size by Laser Diffraction | 575 | nm |
| Ara h ELISA | Ara h1, h2, h3, and h6 present | |
| Protein Content by CBQCA | 7.4 | μg/mg |
| Protein content by Peanut ELISA | 2.2 | μg/mg |
| Burst Release by CBQCA | 30.2% | |
| Particle Size Distribution by SPOS | 520/570/660 | nm |
| (d₁₀/d₅₀/d₉₀) | | |
| Surface protein by Flow Cytometry | 30.3% positive | |
| Endotoxin | <0.048 | EU/mg |

### Example 3. Determining Frequency of TIMP-PPE Particles (CNP-201) with Protein Present on their Surfaces

One vial of CNP-201 was reconstituted in water to form a stable suspension. Similarly, one vial of control particles free from other proteins or peptides (negative control) and one vial of control particles with known high surface protein levels (positive control) were reconstituted in water. Particles were mixed to ensure particles were dispersed homogenously in the suspension.

CNP-201, positive control, and negative control particles were incubated with staining buffer (bovine serum albumin in PBS) containing primary polyclonal anti-peanut protein antibody. A separate set of particles was incubated with staining buffer alone serving as an internal negative control for the experiment. Stained particles were washed 3x in staining buffer by centrifugation and incubated with staining buffer containing fluorophore-conjugated secondary antibody. Particles were then washed 3x in staining buffer.

Stained particles were acquired on a flow cytometer and the frequency of CNP-201 particles staining positive for peanut proteins was compared to the negative and positive control samples. Results were expressed as percentage of particles positive for surface protein when compared to negative control (Table 1 and Table 2).

### Example 4. Pharmaceutical Compositions of TIMP-PPE

Exemplary pharmaceutical formulations or compositions of TIMP-PPE comprise individual components listed in Table 3.

TABLE 3

| Component | 80 g batch | 160 g batch |
|---|---|---|
| PLGA nanoparticles | 30-40% | 30-40% |
| Sucrose | 30-40% | 30-40% |
| Mannitol | 20-30% | 20-30% |
| Sodium citrate | 1-10% | 1-10% |
| Peanut protein | 0.3 μg to 30 μg | 0.3 μg to 30 μg |
| | peanut protein | peanut protein |
| | per mg PLGA | per mg PLGA |

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently, only such limitations as appear in the appended claims should be placed on the disclosure.

### REFERENCES

1. Cannon H E. The economic impact of peanut allergies. Am J Manag Care. 2018; 24 (19 Suppl): S428-s433.
2. Sampath V, Nadeau K C. Newly identified T cell subsets in mechanistic studies of food immunotherapy. J Clin Invest. 2019; 129 (4): 1431-1440.
3. Feuille E, Nowak-Wegrzyn A. Allergen-Specific Immunotherapies for Food Allergy. Allergy Asthma Immunol Res. 2018; 10 (3): 189-206.
4. Chinthrajah R S, Purington N, Andorf S, et al. Sustained outcomes in oral immunotherapy for peanut allergy (POISED study): a large, randomised, double-blind, placebo-controlled, phase 2 study. Lancet. 2019; 394 (10207): 1437-1449.
5. Fleischer D M, Greenhawt M, Sussman G, et al. Effect of Epicutaneous Immunotherapy vs Placebo on Reaction to Peanut Protein Ingestion Among Children With Peanut Allergy: The PEPITES Randomized Clinical Trial. Jama. 2019; 321 (10): 946-955.
6. Vickery B P, Vereda A, Casale T B, et al. AR101 Oral Immunotherapy for Peanut Allergy. N Engl J Med. 2018; 379 (21): 1991-2001.
7. Dunlop J H. Oral immunotherapy for treatment of peanut allergy. J Investig Med. 2020; 68 (6): 1152-1155.
8. Getts D R, Martin A J, McCarthy D P, et al. Microparticles bearing encephalitogenic peptides induce T-cell tolerance and ameliorate experimental autoimmune encephalomyelitis. Nature Biotechnology. 2012; 30 (12): 1217-1224.
9. Hunter Z, McCarthy D P, Yap W T, et al. A biodegradable nanoparticle platform for the induction of antigen-specific immune tolerance for treatment of autoimmune disease. ACS Nano. 2014; 8 (3): 2148-2160.
10. Kelly C P, Murray J A, Leffler D A, et al. TAK-101 Nanoparticles Induce Gluten-Specific Tolerance in Celiac Disease: A Randomized, Double-Blind, Placebo-Controlled Study. Gastroenterology. 2021; 161 (1): 66-80.e68.
11. Prasad S, Neef T, Xu D, et al. Tolerogenic Ag-PLG nanoparticles induce tregs to suppress activated diabetogenic CD4 and CD8 T cells. J Autoimmun. 2018; 89:112-124.
12. Smarr C B, Yap W T, Neef T P, et al. Biodegradable antigen-associated PLG nanoparticles tolerize Th2-mediated allergic airway inflammation pre- and postsensitization. Proc Natl Acad Sci USA. 2016; 113 (18): 5059-5064.
13. Keet C A, Johnson K, Savage J H, Hamilton R G, Wood R A. Evaluation of Ara h2 IgE thresholds in the diagnosis of peanut allergy in a clinical population. J Allergy Clin Immunol Pract. 2013; 1 (1): 101-103.
14. Ozias-Akins P, Breiteneder H. The functional biology of peanut allergens and possible links to their allergenicity. Allergy. 2019; 74 (5): 888-898

1. A method for preparing a composition comprising particles encapsulating peanut proteins, the method comprising:
a. generating a primary emulsion by mixing an aqueous solution of peanut proteins with a solution including a polymer resulting in a primary emulsion;
b. mixing the primary emulsion with a solution including one or more surfactants and/or stabilizers to form a secondary emulsion;
c. hardening the secondary emulsion by evaporation to remove the solvent resulting in hardened polymeric nanoparticles encapsulating peanut proteins within their cores;
d. filtering, washing, and concentrating the nanoparticles; and
e. freeze drying the nanoparticles to form a composition.

2. The method of claim 1, wherein the solution of step (a) includes a solvent or wherein the solution of step (b) includes a solvent, and wherein the solvent is an organic solvent or an inorganic solvent.

3-4. (canceled)

5. The method of claim 1, wherein the solutions of step (a) and step (b) include the same solvent or include different solvents.

6-7. (canceled)

8. The method of claim 1, wherein the emulsion resulting from step (a) or step (b) is a water-in-oil emulsion.

9. (canceled)

10. The method of claim 1, wherein the polymer of step (a) is a biodegradable polymer.

11. The method of claim 10, wherein the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid.

12. The method of claim 1, wherein the surfactant or stabilizer of step (b) is anionic, cationic, or nonionic.

13. (canceled)

14. The method of claim 1, wherein the primary emulsion of step (a) is obtained by homogenization or sonication and/or wherein the secondary emulsion of step (b) is obtained by homogenization or sonication.

15-17. (canceled)

18. The method of claim 1, wherein the pH of the secondary emulsion of step (b) is about pH 4 or less than pH 4.

19-20. (canceled)

21. The method of claim 1, wherein the hardening of nanoparticles in step (c) is performed by evaporation of the solvent.

22-27. (canceled)

28. The method of claim 1, wherein the particles have a negative zeta potential.

29. The method of claim 28, wherein the zeta potential of the particles is between about 0 and −100 mV, or between about −30 and −80 mV.

30. (canceled)

31. The method of claim 1, wherein the particles have a diameter of between about 0.3 μm to 3 μm, between about 0.3 μm to 1 μm, between about 0.4 μm to 1 μm, or between about 0.4 μm to 1 μm.

32-33. (canceled)

34. The method of claim 1, wherein
i) at least 90% of the particles have a diameter of between about 0.3 μm to 3 μm, between about 0.3 μm to 1 μm or between about 0.4 μm to 1 μm;
Ii) wherein at least 50% of the particles have a diameter of between about 0.3 μm to 3 μm, between about 0.3 μm to 1 μm or between about 0.4 μm to 1 μm; or
iii) wherein at least 10% of the particles have a diameter of between about 0.3 μm to 3 μm or between about 0.3 μm to 1 μm.

35-41. (canceled)

42. The method of claim 1, wherein the peanut protein content encapsulated within the particle composition is about 0.1 to 100 μg/mg.

43. The method of claim 1, wherein the peanut proteins comprise Ara h proteins.

44. The method of claim 43, wherein the Ara h proteins are Ara h 1, Ara h 2, Ara h 3, Ara h 4, Ara h 5, Ara h6, Ara h 7, Ara h 8, Ara h 9, Ara h 10, Ara h 11, Ara h 12, Ara h 13, Ara h 14, Ara h15, Ara h 16, Ara h 17 and Ara h 18.

45-53. (canceled)

54. A particle encapsulating peanut proteins made by the method of claim 1.

55. A composition comprising particles encapsulating peanut proteins made by the method of claim 1.

56. The composition of claim 55, further comprising a pharmaceutically acceptable carrier, diluent or excipient.

57. The composition of claim 56, wherein the excipients are sucrose, mannitol, and sodium citrate.

58. A pharmaceutical composition comprising negatively charged particles encapsulating peanut proteins, sucrose, mannitol, and sodium citrate.

59. A method of treating a subject having peanut allergy comprising administering to the subject a particle of claim 54.


SEQUENCE LISTING (25 sequences)

SEQ ID NO: 1 - protein, 619 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRRALILTRAMHSESHPFHFLHLDDMTPELRVAKSHAVNTPGQFEDFFPASSRDQSSYLQGFSRNTLEAAFNAEFNEIRRVLLEENAGGEQEERGQRRWSTRSSENNEGVIVEVSKEHVEELTKHAKSVSKKGSEEEGDITNPINLREGEPDLSDNFGRLFEVKPDKKNPQLQDLDMMLTCVEIKEGALMLPHFNSKAMVIVVINKGTGNLELVAVRKEQQQRGRREQEWEEEEEDEEEEGSNREVRRYTARLKEGDVFIMPAAHPVAINASSELHLLGFGINAENNHRIFLAGDKDNVVDQIEKQAKDLAFPGSGEQVEKLIKNQRESHFVSARPQSQSPSSPEKEDQEEENQGGKGPLLSILKAFN

SEQ ID NO: 2 - protein, 160 aa, Arachis hypogaea
MAKLTILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 3 - protein, 512 aa, Arachis hypogaea
MAKLLALSLCFCVLVLGASSVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 4 - protein, 507 aa, Arachis hypogaea
RQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRDSHQKVHRFDEGDLIAVPTGVAFWLYNDHDTDVVAVSLTDTNNNDNQLDQFPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 5 - protein, 530 aa, Arachis hypogaea
MAKLLELSFCFCFLVLGASSISFRQQPEENACQFQRLNAQRPDNRIESEGGYIETWNPNNQEFECAGVALSRLVLRRNALRRPFYSNAPQEIFIQQGRGYFGLIFPGCPSTYEEPAQQGRRYQSQRPPRRLQEEDQSQQQQDSHQKVHRFNEGDLIAVPTGVAFWLYNDHDTDVVAVSLTDTNNNDNQLDQFPRRFNLAGNHEQEFLRYQQQSRQSRRRSLPYSPYSPHSRPRREEREFRPRGQHSRRERAGQEEEDEGGNIFSGFTPEFLEQAFQVDDRQIVQNLWGENESEEEGAIVTVRGGLRILSPDGTRGADEEEEYDEDQYEYHEQDGRRGRGSRGGGNGIEETICTACVKKNIGGNRSPHIYDPQRWFTQNCHDLNLLILRWLGLSAEYGNLYRNALFVPHYNTNAHSIIYALRGRAHVQVVDSNGNRVYDEELQEGHVLVVPQNFAVAGKSQSENFEYVAFKTDSRPSIANFAGENSFIDNLPEEVVANSYGLPREQARQLKNNNPFKFFVPPFQQSPRAVA

SEQ ID NO: 6 - protein, 131 aa, Arachis hypogaea
MSWQTYVDDHLLCEIEGNHLSSAAILGQDGSVWAQSSNFPQFKPEEITAIMNDFAEPGSLAPTGLYLGGTKYMVIQGEPGTVIRGKKGPGGVTIKKTNQALIIGIYDEPMTPGQCNMIVEKLGDYLIDTGL

SEQ ID NO: 7 - protein, 145 aa, Arachis hypogaea
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 8 - protein, 164 aa, Arachis hypogaea
MVKLSILVALLGALLVVASATRWDPDRGSRGSRWDAPSRGDDQCQRQLQRANLRPCEEHIRQRVEKEQEQEQDEYPYIQRGSRGQRPGESDEDQEQRCCNELNRFQNNQRCMCQALQQILQNQSFRFQQDRSQLHQMERELRNLPQNCGFRSPSRCDLSSRTPY

SEQ ID NO: 9 - protein, 157 aa, Arachis hypogaea
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

SEQ ID NO: 10 - protein, 116 aa, Arachis hypogaea
MASLKFAFVMLVCMAMVGAPMVNAISCGQVNSALAPCIPFLTKGGAPPPACCSGVRGLLGALRTTADRQAACNCLKAAAGSLRGLNQGNAAALPGRCGVSIPYKISTSTNCATIKF

SEQ ID NO: 11 - protein, 92 aa, Arachis hypogaea
LSCGQVNSALAPCITFLTKGGVPSGPCCSGVRGLLGAAKTTADRQAACNCLKAAAGSLHGLNQGNAAALPGRCGVSIPYKISTSTNCATIKF

SEQ ID NO: 12 - protein, 169 aa, Arachis hypogaea
MTDRTQPHTVQVHTTAGRFGDTAAGTNRYPDRGPSTSKVIAVITGLPIGGTLLLFAGLALAGTLLGLAVTTPLFILFSPVIVPAIIVVGLSVAGFLTSGACGLTGLSSFSWVMNYIRQTHGSVPEQLEMAKHRMADVAGYVGQKTKDVGQKTKEVGQEIQTKAQDSKRT

SEQ ID NO: 13 - protein, 150 aa, Arachis hypogaea
MTDRTQPHAVQVHTTAGRFGDTAAGTNRYADRGPSTSKVIAVITGLPIGGTLLLFAGLALAGTLLGLAVTTPLFILFSPVIVPATIVVGLSVAGFLTSGACGLTGLSSFSWVMNYIRQTHGSVPEQLEMAKHRMADVAGYVGQKTKDVGQ

SEQ ID NO: 14 - protein, 137 aa, Arachis hypogaea
MAEALYYGGRQRQEQPRSTQLVKATTAVVAGGSLLILAGLVLAGTVIGLTTITPLFVIFSPVLVPAVITVALLGLGFLASGGFGVAAITVLTWIYRYVTGKHPPGANQLDTARHKLMGKAREIKDFGQQQTSGAQAS

SEQ ID NO: 15 - protein, 137 aa, Arachis hypogaea
MAEALYYGGRQRQDQPRSTQLVKATTAVVAGGSLLILAGLVLAATVIGLTTITPLFVIFSPVLVPAVITVALLGLGFLASGGFGVAAITVLTWIYRYVTGKHPPGANQLDTARHKLMSKAREIKDYGQQQTSGAQAS

SEQ ID NO: 16 - protein, 71 aa, Arachis hypogaea
KTVAGFCIFFLVLFLAQEGVVKTEAKLCNHLADTYRGPCFTNASCDDHCKNKEHFVSGTCMKMACWCAHNC

SEQ ID NO: 17 - protein, 79 aa, Arachis hypogaea
VQKRTIIMEKKMAGFCIFFLILFLAQEYGVEGKECLNLSDKFKGPCLGSKNCDHHCRDIEHLLSGVCRDDFRCWCNRKC

SEQ ID NO: 18 - protein, 72 aa, Arachis hypogaea
MEKKMAGFCIFFLVLFLAQEYGVEGKVCLNLSDKFKGPCLGTKNCDHHCRDIEHLLSGVCRDDFRCWCNRNC

SEQ ID NO: 19 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFTFFSPVIVPAVVTIGLAVTGILTAGACGLTGLMSLSWMINFIRQVHGTTVPDQLDSVKRRMADMADYVGQKTKDAGQQIQTKAQDVKRSSS

SEQ ID NO: 20 - protein, 176 aa, Arachis hypogaea
MATATDRAPHQVQVHTPTTQRVDVQRRGYDVSGGGVKTFLPDRGPSTSQIIAVLVGVPTGGTLLLLSGLSLLGTIIGLAIATPVFTFFSPVIVPAVVTIGLAVIGILTAGACGLTGLMSLSWMINFIRQVHGTTVPDQLDSAKRRMADMADYVGQKTKDAGQEIQTKAQDVKRSSS

SEQ ID NO: 21 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFIFFSPVIVPAVVTIGLAVTGILTAGACGLTGLMSLSWMINFIRQVHGTTVPDQLDSVKRRMADMADYVGQKTKDAGQEIQTKAQDVKRSSS

SEQ ID NO: 22 - protein, 166 aa, Arachis hypogaea
MSDQTRTGYGGGGSYGSSYGGGGTYGSSYGTSYDPSTNQPIRQAIKFMTASTIGVSFLILSGLILTGTVIGLIIATPLLVIFSPILVPAAITLALAAGGFLFSGGCGVAAIAALSWLYSYVTGKHPAGSDRLDYAKGVIADKARDVKDRAKDYAGAGRAQEGTPGY

SEQ ID NO: 23 - protein, 68 aa, Arachis hypogaea
VTCTPTELSPCLGAITGGSPPSSVCCQKLRAQKPCLCNYIKNPALRTYVNSPGARRVASSCGVPLPSC

SEQ ID NO: 24 - protein, 93 aa, Arachis hypogaea
AIQCSFVTKSIAPCFGYLKSGGTVSGPCCSGIQNINGTAKTTSDRQAVCNCLKSAAASLGSQINPNNAASLPGKCGVSIPYKISTSINCSSIK

SEQ ID NO: 25 - protein, 226 aa, Arachis hypogaea
MANPRVFFDMTIGGQPAGRIIMELFADTTPRTAENFRALCTGEKGVGRSGKPLHYKGSSFHRVIPNFMCQGGDFTAGNGTGGESIYGSKFADENFIKKHTGPGILSMANAGQNTNGSQFFICTAKTEWLDGKHVVFGQVVEGLDVVREIEKVGSSSGRTSKPVVVADCGQLCNAKLTMSWGNGAYRKSLQVQGVVIGQRDANSMSEQAQFAFVVSFVTQIMKIQSS